SEQUENCE LISTING (8 sequences)

SEQ ID NO: 1 - DNA, 388 bp, Grapevine fanleaf virus
cataagttgggcacgttttcatgtgaaatagactatggagaattgtgtggtcatgctatgtggtttaagtccacaacatttgaatctccgagactacattttacgtgtttaacgggcaacaacaaagagctagcggcagactggcaagctgtcgtcgagctatatgctgaattggaagaggctaccacttttcttgggaagccaactttggtttttgatccaggtgtttttaatggcaaattccaatttcttacttgccctcccatattttttgacctaacagccgtcacggctcttaagagtgctgggctgaccttgggacaagttccgatggttggtactaccagagtttataacctaaatagcactcttgtgagttgtgttttgg

SEQ ID NO: 2 - DNA, 388 bp, Grapevine fanleaf virus
ccaaaacacaactcacaagagtgctatttaggttataaactctggtagtaccaaccatcggaacttgtcccaaggtcagcccagcactcttaagagccgtgacggctgttaggtcaaaaaatatgggagggcaagtaagaaattggaatttgccattaaaaacacctggatcaaaaaccaaagttggcttcccaagaaaagtggtagcctcttccaattcagcatatagctcgacgacagcttgccagtctgccgctagctctttgttgttgcccgttaaacacgtaaaatgtagtctcggagattcaaatgttgtggacttaaaccacatagcatgaccacacaattctccatagtctatttcacatgaaaacgtgcccaacttatg

SEQ ID NO: 3 - DNA, 1346 bp, Cauliflower mosaic virus
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacg

SEQ ID NO: 4 - DNA, 742 bp, Flaveria trinervia
tcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattg

SEQ ID NO: 5 - DNA, 766 bp, Agrobacterium tumefaciens
ctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaat

SEQ ID NO: 6 - DNA, 233 bp, Agrobacterium tumefaciens
gggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatcc

SEQ ID NO: 7 - DNA, 946 bp, Artificial Sequence
  Transposon Tn5
gggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccc

SEQ ID NO: 8 - DNA, 706 bp, Agrobacterium tumefaciens
gatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

(19) United States
(12) Patent Application Publication
(10) Pub. No.: US 2015/0067916 A1
(43) Pub. Date: Mar. 5, 2015

(54) GENERATION OF GRAPEVINE ROOTSTOCKS THAT PROVIDE RESISTANCE AND SANITATION IN RELATION TO GRAPEVINE FANLEAF VIRUS (GFLV)

(71) Applicant: PONTIFCIA UNIVERSIDAD CATOLICA DE CHILE, Santiago (CL)

(72) Inventors: Patricio Arce Johnson, Santiago (CL); Elizabeth Torres Pacheco, Santiago (CL); Francisca Godoy Santin, Santiago (CL); Consuelo Medina Arevalo, Santiago (CL); Agnes Cadavid Labrada, Santiago (CL); Pablo Valenzuela Valdes, Santiago (CL); Consuelo Bruno Urbina, Santiago (CL)

(21) Appl. No.: 14/358,884

(22) PCT Filed: Nov. 9, 2012
(86) PCT No.: PCT/IB2012/056297

§ 371 Date: May 16, 2014

(30) Foreign Application Priority Data
Nov. 16, 2011 (CL) 2884-2011

(51) Int. Cl. C12N 15/82

(52) U.S. Cl. CPC C12N 15/8283
USPC 800/279; 435/320.1; 435/419

(57) ABSTRACT

The invention discloses a vector plasmid called “GFLV silencing construct” that confers resistance and sanitation against the grapevine fanleaf virus (GFLV); plant cells transformed with said vector plasmid and a method to impart resistance and sanitation against the grapevine fanleaf virus (GFLV) in non-transgenic grapevines when being grafted onto seedlings generated from cells transformed with said plasmid vector. The “GFLV silencing construct” plasmid vector of the invention comprises inverted sequence duplicates coding for the grapevine fanleaf virus (GFLV) capsid protein.

### OBJECT OF THE INVENTION

This invention discloses a vector plasmid called “GFLV silencing construct” that confers resistance and sanitation against the grapevine fanleaf virus (GFLV); plant cells transformed with such plasmid vector and a method to confer resistance and sanitation against the grapevine fanleaf virus (GFLV) to non-transgenic grapevines when grafted in seedlings generated from cells transformed with said plasmid vector.

### BACKGROUND OF THE INVENTION

The fanleaf disease, caused by the grapevine fanleaf virus (GFLV), is one of the most severe and devastating grapevine diseases. Sensitive cultivars show a fast decay, low fruit quality and yield decrease. Affected grapevines have a lower size when compared to healthy grapevines. More than 80% of production loss can be expected in severe infections. The longevity of the vineyard considerably decreases in sensitive varieties. Infected grapevines show deformed leaves with the appearance of an open fan (this is the reason why the disease is called “fanleaf” disease). Other visible symptoms include leaf yellowing (mosaic), clear yellow bands close to veins (“vein banding”), abnormal branching and short internodes. Grapevines affected by fanleaf disease have poor fruit setting and heterogeneous fruit ripening. Grapevine fanleaf virus is transmitted by the sting of the nematode Xiphinema index. This disease is found in all areas where Vitis vinifera and American rootstock hybrids are cultivated.

The grapevine (Vitis vinifera L.) is one of the main fruit producing species cultivated in Chile, both for wine production and for table grapes, with a cultivated surface destined for exploitation of this species is approximately 135,775 hectares.

Among the main phytosanitary problems in grapevine production in Chile are nematodes of genus Xiphinema, or dagger nematodes, which cause root and viral problems. The species that are most frequently found are Xiphinema index and Xiphinema americanum sensu lato. Thus, in the central zone of the country (Metropolitan Area), near 70% of the examined grapevine plantation parcels showed some degree of infestation, between slight and very high, with both Xiphinema species, being common the presence of high populations in other wine and grape producing regions of the north, center and south of the country. These Xiphinema species directly damage roots, causing deformations, sometimes galls and generally less vigor in affected plants. However, their major importance resides in them being vectors of relevant nepoviruses. As described before, Xiphinema index transmits the fanleaf virus, the most economically important nepovirus in grapevines.

There are several options for the control of fanleaf disease. The most used strategy is altering the biological cycle of the nematode-virus relationship, for instance through the control of nematode reservoir weeds or by eradication of the nematode through fumigation with nematicides. Traditionally, chemical nematicides are used, which currently are highly questioned products due to their adverse effects for organisms and agricultural ecosystems and their high cost. Therefore, it is necessary to develop other control alternatives that are ecologically benign and sustainable.

At present, there are also commercially available grapevine rootstocks resistant to ground nematodes and therefore have a better behavior against virus infection. However, these rootstocks can be infected when virus carrying hardwood cuttings are grafted onto them. Hence, the most suitable way to avoid the disease would be to establish vineyards resistant to GFLV. Under these conditions, plants would be homogeneous in fruit productivity and quality.

### STATE OF THE ART

As described above, one of the strategies assayed to avoid damages directly caused by nematodes or by infections transmitted by them, such as the grapevine fanleaf virus (GFLV) infection has been the development of nematode resistant rootstocks. Among the best known nematode resistant rootstocks are Couderc 1613, Dogridge and Harmony, but it has been reported that these rootstocks do not provide an adequate protection against these organisms and hence against pathogens transmitted by them, such as the grapevine fanleaf virus (GFLV). Moreover, the use of these nematode resistant rootstocks would not eliminate a preexistent infection in grafted plants, since they do not have any type of resistance and sanitation against the grapevine fanleaf virus (GFLV) by themselves.

A second approach to avoid damages caused by phytopathogenic viruses, such as the grapevine fanleaf virus (GFLV), has been the generation of plants having a direct resistance against said viruses and not for the transmitting nematode. Documents that address the technical problem through this approach describe plasmids that confer resistance against the phytopathogen when expressed in a plant cell. The transformed cell can be part of a transformed plant or a transformed rootstock, being this second alternative the most convenient one since the final product, i.e. the grape, is not transgenic. This is the strategy used in the present invention, which also has the enormous advantage of being able to sanitize grafted infected plants. In what follows, we will analyze the documents that use this same approach and are closer to the present invention.

The Chilean national application CL 01837-2003, belonging to the same inventors of the present invention, is directed to a DNA construct formed by a vector plasmid containing repeated inverted DNA sequences that are identical in at least 23 nucleotides to the RNA of the grapevine fanleaf virus (GFLV); and a procedure to produce commercial variety grapevines that are made resistant through the introduction of said DNA construction, thus obtaining a transgenic GFLV-resistant grapevine plant. On the other hand, the present invention is directed to a transgenic rootstock onto which a non-transgenic grapevine will be inserted, to which the rootstock will confer resistance and sanitation. The mechanism used in the present invention consists in a construct with a vector containing a specific 388 bp fragment of the gene coding for a fragment of the GFLV coat protein, both sense and antisense, which will form a dsRNA hairpin. This triggers the plant silencing system, which produces the specific degradation of the gene coding for the coat protein, thus preventing virus assembly.

The application CL 02069-1995 was granted on Jul. 21 2004 under the registry number CL 42208 and refers to a DNA coding for the NIA protein of the type W FLA83 strain of the papaya ringspot virus (PRV), vectors and cells containing said DNA and a method to produce plants resistant to the PRV through transformation of plants with NIA. This patent is directed to a pathogen that is different from the pathogen of the present invention, discloses a DNA molecule that codes for the NIA protease of the PRV virus and teaches the production of virus resistant plants. The present invention discloses a transgene that codes for a 388 bp fragment of the GFLV coat protein, both sense and antisense, spaced apart by a PDK intron, under control of the CaMV35S promoter. Furthermore, the invention discloses the transformation of somatic embryos of the 110Richter Vitis vinifera rootstock and the regeneration of transgenic rootstocks by somatic embryogenesis, which is not described in Patent CL 42208.

The applications US/2007 7211710 and US/2003 6667426 teach a method to produce and select transgenic grapevine plants or grapevine component (e.g. an embryo, graft or rootstock) resistant to the grapevine fanleaf virus (GFLV) disease. These documents are directed to a method comprising the following steps: a) the transformation of grapevine cells with the sequence of the coat protein or a fragment thereof (preferably having 40-80 nucleotides or more), which is capable to be expressed in the cell, b) the regeneration of transgenic grapevines or grapevine components and c) the selection of transgenic grapevines or grapevine components that express low levels of nucleic acid molecules, which increase resistance against the disease. The main difference between these documents and the present invention is that the construct used in US/2007 7211710 and US/2003 6667426 contains the complete sequence of the GFLV coat protein or fragments thereof, sense or antisense, which are expressed in transformed cells. The construct of this invention contains a 388 bp fragment of the coat protein both sense and antisense spaced apart by an intron. This construct does not express the protein but expresses a double stranded RNA that triggers the gene silencing of the virus coat protein and thus induces resistance and sanitation against the disease. Furthermore, the present invention uses a graft on a transgenic graft that expresses the transgene and transmits resistance and sanitation to the graft. In this way, the desired grapevine variety is not transgenic, although it has resistance and sanitation against the disease.

The Argentinian Patent AR023987(A1) shows a method for the production of grapevine somatic embryos with resistance and sanitation against a pathogen by culturing a somatic grapevine embryo in a medium containing a growth regulator and a filtered pathogen culture, without including transforming plant cells with a plasmid. On the contrary, the present invention includes the genetic transformation of the somatic embryo with a transgene coding for a pathogen genome fragment, by means of what a transgenic plant resistant to the virus is obtained. This is used as a rootstock onto which different virus susceptible varieties of grapevine are grafted and conferred induced resistance and sanitation.

The control of the fanleaf disease, caused by the GFLV, mainly consists in an adequate control of the transmitting nematode, X. index. However, ground fumigation is difficult and gives good results only few times. Other practices, such as culture rotation, direct plantation and weed control are equally little effective. Therefore, the local control of virus propagation based on nematode control is difficult to restrict. On the contrary, long distance virus propagation can be controlled through the production and distribution of healthy propagation material (buds, rooted rootstocks and grafted plants). The virus can be eliminated through micrografting, in vitro meristem culture (virus-free sections) and/or thermotherapy, currently the most used procedure. In general, thermotherapy consists in the treatment of cultures of small apical meristems (in vitro thermotherapy) or seedlings (in vivo thermotherapy) in growth chambers at high temperatures (around 37° C.), and then taking the apical segments and transfer them to culture media to allow their elongation and rooting. In this way, virus-free seedlings can be regenerated, based on the fact that high temperatures inhibit viral propagation.

These methods allow the production of virus-free propagation material, but have the disadvantage of being slow and laborious and not being able to heal the disease in productive plants, since it produces seedlings that will take years to bear fruit. This procedure for grapevines has an efficiency not higher than 60% and takes between 4 and 6 months of treatment. The method of this invention allows a non-transgenic productive graft, healthy or infected, to acquire resistance and sanitation when grafted onto the transgenic rootstocks of this invention and therefore remains 100% sanitized from virus presence. We have observed that using this procedure applied with the rootstocks of the invention, 100% sanitation is obtained after a month from grafting in greenhouse conditions.

According to the previous discussion, the present invention, consisting of a plasmid vector that comprises inverted sequence duplicates coding for the grapevine fanleaf virus (GFLV) coat protein; and a procedure to produce a grapevine rootstock resistant against the grapevine fanleaf virus (GFLV) by means of the introduction of said vector plasmid; has not been anticipated by the previous art and solves the technical problem of imparting resistance and sanitation against the GFLV to grapevine plants.

### BRIEF DESCRIPTION OF THE INVENTION

The invention discloses a vector plasmid called “GFLV silencing construct” that confers resistance and sanitation against the grapevine fanleaf virus (GFLV); plant cells transformed with said vector plasmid and a method to impart resistance and sanitation against the grapevine fanleaf virus (GFLV) in non-transgenic grapevines when being grafted onto seedlings generated from cells transformed with said plasmid vector.

The “GFLV silencing construct” plasmid vector of the invention comprises inverted sequence duplicates coding for the grapevine fanleaf virus (GFLV) coat protein.

To achieve the goal of conferring resistance and sanitation to grapevines against GFLV, we have used a strategy based on the mechanism of interference RNA (iRNA). In broad terms, when a double stranded RNA (dsRNA) enters into the cell, this is recognized by the Dicer enzyme, which cuts this dsRNA in fragments having 21 to 25 nucleotides (siRNA). These siRNA fragments bind the RISC complex that promotes the degradation of any sequence homologous to that of the involved siRNA. Based on this, we have created vectors into which DNA sequences are cloned as inverted duplicates. Then, when transcribed, these form a dsRNA hairpin that will trigger the iRNA system. In this case, the vector plasmid used in this invention contains inverted duplicates with sequences coding for the GFLV coat protein, which are spaced apart by an intron and together will form the hairpin that, as previously described, will promote the specific degradation of the viral gene coding for the virus coat protein. This plasmid is introduced in the genome of the plant through any transformation technique available in the art, which, in a preferred embodiment, is a genetic transformation mediated by the bacteria Agrobacterium tumefaciens.

With this strategy, GFLV resistant transgenic lines are obtained. These transgenic lines are used as a rootstock for grafts, into which the silencing signal is propagated through the phloem. These grafts can be of any variety with a commercial interest, both for table grape and wine grape production. In this way, this invention offers a method by which the (non-transgenic) plant grafted onto the transgenic rootstock acquires resistance and sanitation against GFLV. This is obtained without the disadvantages of transgenic products for the exporting industry.

We have performed in vitro and greenhouse assays in which we demonstrate by using RT-PCR that when a previously tested GFLV infected plant is grafted onto the GFLV resistant rootstock of the invention, there are no detectable virus transcripts in the plant, specifically those belonging to the virus movement and coat proteins. This indicates that the method of the present invention, which uses the vector plasmid of the invention, achieves total silencing of the GFLV coat protein and thus eliminates the viral infection.

### DESCRIPTION OF FIGURES

FIG. 1 (A): “GFLV silencing construct” vector plasmid, containing 338 bp of the GFLV coat protein (pc-gflv) under control of the 35S promoter of the cauliflower mosaic virus (CaMV 35S) and the nptII gene that confers resistance and sanitation against kanamycin for the selection of transgenic plants.

FIG. 1 (B): double stranded RNA (dsRNA) coming from vector transcription.

FIG. 2: RT-PCR analysis of one of the grafts performed under greenhouse conditions, using my-GFLV primers. The first lane contains cDNA from graft i28/1, the second lane contains the RT control for the same i28/1 graft (RT−), the third lane contains cDNA from the infection-carrying grafted plant (GFLV+) and the last lane contains the negative PCR control.

### DETAILED DESCRIPTION OF THE INVENTION

The present invention is directed to the “GFLV silencing construct” plasmid vector, which comprises inverted sequence duplicates coding for a fragment of the grapevine fanleaf virus (GFLV) coat protein. and a procedure to produce a grapevine rootstock resistant against the grapevine fanleaf virus (GFLV) by means of the introduction of said vector plasmid. In this way, the invention provides a new transgenic grapevine rootstock resistant to GFLV, the main causing agent of the fanleaf disease in Vitis vinifera plantations around the world. Resistance and sanitation are obtained through the expression of a transgene containing 388 bp of the sequence of the virus coat protein, both sense and antisense and spaced apart by an intron. In this way, a double stranded RNA molecule is generated when this transgene is transcribed, which triggers the natural plant silencing system. With this strategy, any complementary double stranded RNA in the cell is degraded, thus preventing virus packing and systemic propagation. On the other hand, the silencing signal can be propagated through the phloem and can then movement towards the grafts, where it will induce resistance and sanitation without requiring those grafts to be transgenic. Thus, virus-free fruit is obtained with high market competitiveness.

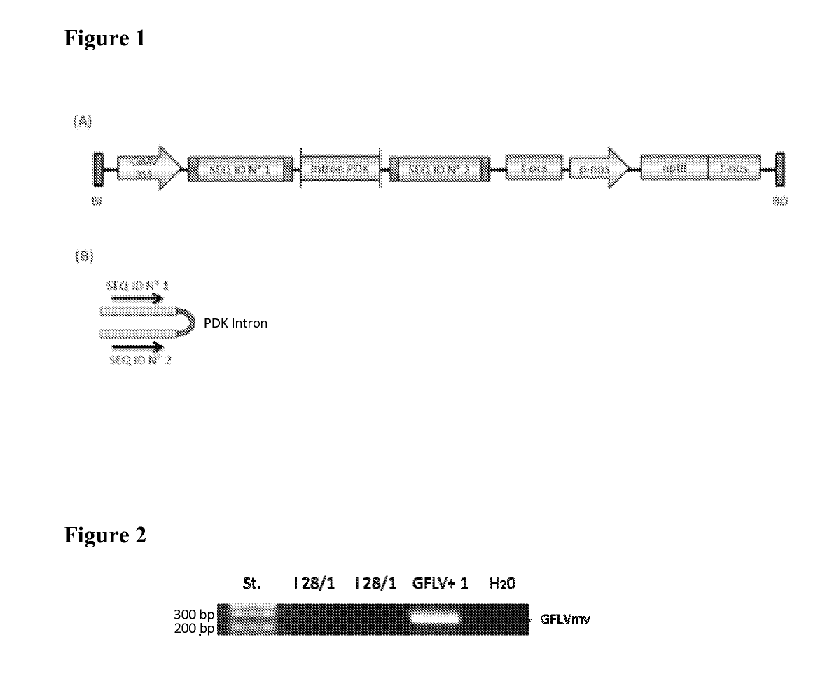

Firstly, the inventors developed a new vector plasmid (see FIG. 1(A)), the expression of which in a plant cell confers resistance and sanitation against GFLV to said plant cell. As mentioned above, the strategy used to design the plasmid consists in inducing the iRNA system in the plant. Initially, 3 constructions were tested, with 3 different fragments of the viral genome: the first fragment consisted in 260 bp of the sequence involved in RNA2 replication, the second sequence contained 340 bp coding part of the virus movement protein, and the third fragment had 388 bp of the virus coat protein gene. 3 vectors were obtained (pRNAiR, pRNAiMP and pRNAiCP) from the genome of the Chilean strain of GFLV and 3 transgenic lines were generated with the 3 constructs.

Finally, the third construct consisting in a 388 bp fragment of the gene that codes for the GFLV coat protein (cp-gflv) was selected (SEQ ID NO: 1) and was introduced in the vector plasmid in sense direction (SEQ ID NO: 2) in the plasmid vector. This was done since the coat virus protein transcript is the more abundant, and in this way the virus assembly can be prevented.

The plasmid pHellsgate2 was used as a base, which allows the introduction of a sense and antisense transgene inside the vector, spaced apart by a spacer region, in this case a PDK intron. This does not fulfill any part in the post-transcriptional gene silencing (PTGS), but stabilizes the pHellsgate vector and significantly increases the silencing efficiency. In this way, hairpins will form when RNA is transcribed (see FIG. 1 (B)), which will induce the iRNA system.

The pHellsgate2 vector plasmid also comprises a CaMV35S promoter, and an ocs terminator, which are operatively linked to the sense transgene-PDK intron-antisense transgene complex. It also comprises the neomycin phosphotransferase II gene (nptII) that comprises resistance and sanitation against the antibiotic kanamycin, which is useful for selection during transformation and regeneration. The plasmid of the invention is shown in FIG. 1 (A) and the hairpin formed when the sense transgene-PDK intron-antisense transgene is transcribed is shown in FIG. 1 (B).

The invention was created based on the Chilean GFLV strain, known as Ch-80, which shares 90% identity with other virus strains from other parts of the world. As the system is designed for this particular strain, it is highly specific. As mentioned above, we selected the coat protein sequence as silencing target, since it is the most abundant viral transcript and this makes it a good silencing target. Thus, virus assembly is prevented. From this transcript, 388 bp of the 5′ region of the gene were selected, since in this region silencing is highly efficient and this length forms very stable dsRNAs.

Agrobacterium tumefaciens was transformed with the vector plasmid of the invention and transformed Agrobacterium tumefaciens is used to transform embryogenic grapevine calluses. Preferably, the 110Richter grapevine rootstock is used, but any other rootstock can be used, such as Freedom and Harmony, which have been successfully tested in our laboratory. Both transformations can be performed using any known protocol in the state of the art without altering the result of the invention.

Transformed calluses are differentiated into rootstocks with traditional differentiation methods to obtain the rootstocks of the invention. The inventors have surprisingly demonstrated that when plants infected with GFLV are grafted onto these rootstocks, the infection was eliminated after a time lapse as short as 4-5 weeks, and no disease reappearance was observed when evaluating the plants 6 months later. This is demonstrated in Example 8 of the invention.

Therefore, when any non-transgenic Vitis vinifera variety is grafted onto a rootstock transformed with the “GFLV silencing construct” vector plasmid of the invention, it acquires resistance against GFLV. Among Vitis vinifera varieties that can be grafted, we can mention, for example: Autumn royal, Black seedless, Calmeria, Emperor, Flame seedless, Loose Perlette, Red Malaga, Ruby seedless, Loose Perlette, Thompson seedless, Red Globe, Sugarone and Superior seedless, among table grape varieties; and Carmenère, Cabernet sauvignon, Cabernet Franc, Syrah, Chardonnay, Courdec, Dattier, Emerald, Malbec, Merlot, Mission, Muscat, Pinot noir, Riesling, Sauvignon blanc, Sémillon, Shiraz, Tempranillo, Zinfandel, among wine grape varieties. It should be pointed out that any variety already commercially available or any other that could be developed in time are susceptible of being grafted onto the rootstocks of the present invention to sanitize them or confer them resistance against GFLV.

Surprisingly, the inventors have found that the silencing signal movements through the phloem from the transformed rootstock to the non-transformed plant and, in this way, the grafted plant acquires resistance and sanitation against the grapevine fanleaf virus (GFLV) without being transformed. This avoids plants to be infected by the virus or, as mentioned above, allows infected plants grafted onto the transformed rootstock of the invention to recover from infection.

In a preferred embodiment, the invention is used to transform the 110Richter rootstock. This rootstock is used to graft wine and table grapes and it was therefore selected to develop a regeneration and transformation system for resistance and sanitation against the virus. The present invention describes for the first time the regeneration and transformation for this resistance and sanitation feature that has a large importance for the winemaking and fruit industry. Novel aspects of this invention are represented by:
Used virus strain. The invention was created based on the Chilean GFLV strain, known as Ch-80, which shares 90% identity with other virus strains from other parts of the world. As the system is designed for this particular strain, it is highly specific.
Considered viral RNA sequence. Likewise, we selected RNA sequence coding for the coat protein as silencing target, since it is the most abundant RNA transcript in the infected cell, which makes it an excellent target to induce viral gene silencing. In this way, virus assembly and subsequent virus dissemination to the different plant tissues and to other plants through the X. index vector is prevented.
The size of the sequence incorporated into the vectors. We selected 388 bp from the transcript of the region corresponding to the 3′ segment of the gene that codes for the coat protein. This selection also constitutes an innovative element since the selected region will induce much more efficiently the gene silencing than when considering the central or 5′-end regions of the gene. In this way, longer and more stable dsRNA will be generated to be degraded by the silencing machinery of the cell. It has been observed that shorter sequences are less stable and makes the system less efficient.
Resistance and sanitation method against the virus. Another relevant and novel aspect in which the inventive level of the invention is observed is related to the mechanism of infection of this virus in the plant and therefore the resistance and sanitation implemented in this invention. The GFLV is transmitted by the nematode X. index, which acquires the virus when feeding from an infected plant and transmits it to a healthy plant when feeding from it. Since this invention considers the expression of this dsRNA that triggers the resistance and sanitation response in the roots of the 110Richter rootstock, the resistance and sanitation response will be immediately induced when the virus arrives to the root cell by means of the X. index nematode feeding, preventing the virus to disseminate to other root cells and through the vascular tissue to all the plant. For this reason, the generated transgenic rootstocks, besides sanitizing infected plants, will be able to avoid viral infection in healthy plants, thus ensuring good plant development and fruit production, being this the first case of generation of resistant grapevines using this methodology.

The information described in the following examples is merely illustrative of the present invention, since there are other embodiments that fall within the scope of the present invention.

### Examples

### 1. Plant Material and Establishment of Embryogenic Cultures

An embryogenic culture was established from anthers and ovaries extracted from immature inflorescences of the grapevine 110Richter rootstock, between 13-15 days before anthesis. This is the first work describing the use of this important rootstock for the winemaking and table grape industry for regeneration and transformation into resistance and sanitation against viruses.

Inflorescences were wrapped in wet paper towels and placed in refrigeration for transportation. Once in the lab, they were washed with tap water and placed in Parafilm-sealed Petri dishes at 4° C. for 48 hrs. Subsequently, they were cut in small clusters and disinfected in a 20% by volume sodium hypochlorite solution with three drops of 1% by volume Tween 20, under constant stirring for 10 min. Then they were rinsed four times with distilled water for 3 min each time.

Sterile inflorescences were placed in Petri dishes and each flower was dissected under a stereomicroscope. With the help of sharp-pointed dissection tweezers and a scalpel, calyptrae were removed from flowers and anthers together with their filaments and ovaries were isolated. Subsequently, 300 anthers were cultured in Petri dishes with 25 mL of PIV medium (Table 1) for a period of 7 months with monthly subcultures. 20 anthers were cultured per dish. Cultures were kept in a growing chamber at a temperature of 25° C.±1° C., in the dark, until embryogenic tissue was obtained.

TABLE 1
Culture media

| | Culture media concentrations | | | |
|---|---|---|---|---|
| Composition | PIV | DE | GE | GS1CA |
| Macronutrients (mg · L⁻¹) | | | | |
| KNO₃ | 950 | 950 | 950 | 950 |
| NH₄NO₃ | 720 | 825 | 825 | 720 |
| CaCl₂ | 166 | 166.1 | 166.1 | 166 |
| MgSO₄ | 90.37 | 90.35 | 90.35 | 90.37 |
| KH₂PO₄ | 68 | 85 | 85 | 68 |
| Micronutrients (mg · L⁻¹) | | | | |
| MnSO₄•H₂O | 18.9 | 16.9 | 16.9 | 18.9 |
| H₃BO₃ | 10 | 6.2 | 6.2 | 10 |
| ZnSO₄•7H₂O | 10 | 8.3 | 8.3 | 10 |
| KI | — | 0.83 | 0.83 | — |
| CuSO₄•5H₂O | 0.025 | 0.025 | 0.025 | 0.025 |
| CoCl₂•6H₂O | — | 0.025 | 0.025 | — |
| Na₂MoO₄•2H₂O | 0.25 | 0.25 | 0.25 | 0.25 |
| FeSO₄•7H₂O | 27.8 | 27.8 | 27.8 | 27.8 |
| Na₂EDTA•2H₂O | 37.26 | 37.26 | 37.26 | 37.26 |
| Vitamins (mg · L⁻¹) | | | | |
| Nicotinic acid | 0.5 | 0.5 | 0.5 | 0.5 |
| Pyridoxine HCl | 0.5 | 0.5 | 0.5 | 0.5 |
| Thiamin HCl | 0.1 | 0.1 | 0.1 | 0.1 |
| Myoinositol | 100 | 100 | 100 | 100 |
| Glycine | 2 | 2 | 2 | 2 |
| Other components | | | | |
| Sucrose (g · L⁻¹) (Merck) | 60 | 30 | 30 | 60 |
| Activated charcoal (g · L⁻¹) (Merck) | — | 2.5 | 2.5 | 2.5 |
| pH | 5.8 | 5.8 | 5.8 | 5.8 |
| Gelrite (g · L⁻¹) (Sigma-Aldrich Co.) | 3 | 5 | 5 | 3 |
| BA (mg · L⁻¹) (Sigma-Aldrich Co.) | 2 | — | — | 0.2 (1 μM) |
| | (8.9 μM) | | | |
| 2.4-D (mg · L⁻¹) (Sigma-Aldrich Co.) | 1 (4.5 μM) | — | — | — |
| IAA (mg · L⁻¹) (Sigma-Aldrich Co.) | — | — | 1.7 (10 μM) | 3.5 (20 μM) |
| GA₃ (mg · L⁻¹) (Sigma-Aldrich Co.) | — | — | 0.35 (1 μM) | — |
| NOA (mg · L⁻¹) (Sigma-Aldrich Co.) | — | — | — | 2 (10 μM) |

### 2. Maintenance and Differentiation of Embryogenic Calluses for Long Periods

Embryogenic cultures were kept in a growing chamber at a temperature of 25° C.±1° C., in the dark. An innovation in the embryogenic tissue culture was the alternation of PIV medium and embryo proliferation medium GS1CA (Table 1) for a two months period in each medium, with monthly subcultures. This procedure alternating between regeneration and differentiation medium allow the generation of large amounts of embryogenic tissue for transformation, contrarily to other described procedures that not consider alternate culture.

25 embryogenic callus clusters of about 1 cm² were selected. Then, 5 clusters were cultured in a Petri dish with 25 mL of embryo differentiation medium DE (Table 1), for one month. Using this procedure, somatic embryos at different developmental stages were obtained: globular, heart and torpedo.

### 3. Plasmids

The binary plasmid pHellsgate2 was used, with modifications in the incorporated viral RNA sequence, which uses an approach based on a series of recombinations for the introduction of a sense and an antisense transgene into the vector. A 388 bp fragment of the gene that codes for the GFLV coat protein (cp-gflv) was chosen, in sense direction (SEQ ID NO: 1) and antisense direction (SEQ ID NO: 2), spaced apart by a PDK intron, under the control of the CaMV35S promoter and the ocs terminator. The plasmid also contains the neomycin phosphotransferase II gene (nptII) that confers resistance against the antibiotic kanamycin (see FIG. 1). This is useful during transformation and regeneration.

### 4. Transformation of Agrobacterium tumefaciens

Competent Agrobacterium tumefaciens strain GV3101 cells were transformed with the pHellsgate2 plasmid containing the construct SEQ ID NO: 1—PDK intron—SEQ ID NO: 2 (see FIG. 2). 100 μL of competent cells stored at −80° C. were thawed and 1 μg of plasmid was then added. They were subsequently frozen in liquid nitrogen for 5 min and thawed at 37° C. for 25 min. Then, 1 mL of LB (10 g/L Tryptone, 5 g/L yeast extract and 10 g/L NaCl) was added, with antibiotics for selection of the GV3101 strain (50 mg/L gentamicin and 10 mg/L rifampicin) and for the pHellsgate2 plasmid (50 mg/L spectinomycin). The culture was grown at 28° C. for 3 hours with stirring and then concentrated to be plated in solid LB medium with antibiotics. Finally, cultures were left at 28° C. for 48 hrs. in the dark and colonies are checked using PCR. For this, we used the following primers:

Primer 5′-GFLV241:
5′-GCTCATAAGTTGGGCACGTT-3′;
Primer 3′-GFLV241:
5′-TGCCATTAAAAACACGTGGA-3′.

These give rise to a 241 bp fragment of the transgene. The PCR reaction consisted in 35 cycles at 94° C. for 50 s, 52° C. for 50 s and 72° C. for 90 s. PCR products were visualized by electrophoresis in 1% agarose gels using 0.5×TAE buffer and stained with ethidium bromide under UV light.

### 5. Transformation and Regeneration of Transgenic Plants

For transformation of the embryogenic calluses, a culture of Agrobacterium tumefaciens strain C58GV3101 harboring the plasmid pHellsgate2 modified with our viral sequence and 14 g of embryogenic calluses from anthers of the 100Richter rootstock with somatic embryos in globular state were used.

Untransformed embryogenic calluses subjected to the same procedure, except that Agrobacterium infection was simulated with distilled sterile water, were used as a transformation control. After one month after Agrobacterium inoculation, no bacterial growth was observed in 36 transformed calluses. The same happened in 36 untransformed embryogenic calluses used as positive controls. After 2 months of culture in selection/induction medium, from 72 transformed and uncontaminated calluses, 39 (54%) showed antibiotic resistance. From these, 19 (48%) grew and kept their embryogenic capacity, with white globular somatic embryos. After one month of culture, from the 19 embryogenic calluses in selection/differentiation medium with kanamycin, globular- and torpedo-shaped embryos were formed together with brown zones that did not show any antibiotic resistance. 200 embryos were isolated in torpedo state. After one month of culture in germination medium with no antibiotics for selection of transgenic plants, 160 embryos (80%) germinated normally and 40 (20%) did aberrantly. 142 complete plants with root formation were obtained.

### 6. Detection of the Transgene

The transgene was detected through PCR. DNA was extracted from leafs of seedlings grown in vitro, following the protocol described by Lodhi et al. (1994). Leaf fragments were placed in Eppendorf tubes and frozen in liquid nitrogen. Then, 1 g of leaves was macerated in a porcelain mortar with liquid nitrogen until a fine powder was obtained. Then, 1 mL of extraction solution at 65° C. (2% w/v CTAB, 1.4 M NaCl, 20 mM EDTA, 100 mM Tris-HCl pH 8.0) with 0.2% v/v β-mercaptoethanol 0.2% and 10 mg/g PVP40 leaf were added. The mix was homogenized and transferred into a 1.5 mL Eppendorf tube, and then incubated at 60° C. for 30 min with mild stirring and centrifuged at 10,000 rpm for 5 min at 4° C. The supernatant was recovered in a new Eppendorf tube and 1 volume chloroform-isoamylic alcohol (24:1) was added, the mix was mildly mixed by inversion and centrifuged at 10,000 rpm for 15 min at 4° C. The aqueous phase was recovered and ½ volume of NaCl and 2 volumes of absolute cold ethanol were added, the mix was mildly homogenized by inversion and DNA was precipitated at −20° C. for 30 min. Then, it was centrifuged at 10,000 rpm for 15 min at 4° C., the supernatant was discarded and the precipitate was washed with cold 70% ethanol and then centrifuged in the same previous conditions. The Eppendorf tube with the DNA precipitate was placed inverted on a sterile paper towel to dry the precipitate at ambient temperature. Subsequently, DNA was suspended in 100 μL distilled water and incubated at 37° C. for 1 hour with 3 μL RNase A. To eliminate the excess of salts and RNase A, a second DNA extraction was performed. 100 additional μL of distilled deionized water were added and the previously described protocol was repeated, except for the addition of NaCl and the incubation with RNase A. The extracted DNA was kept at −20° C. Its concentration was spectrophotometrically determined by diluting 1 μL of DNA in 1 mL and placing it in a 1 cm light path length quartz cuvette. Sample absorbances at 260 nm and 280 nm were determined. One absorbance unit at 260 nm was considered as equivalent to 50 μg/mL of double stranded DNA, and thus DNA concentration was estimated by using the formula [DNA] (μg/mL)=A₂₆₀ₙₘ×50×dilution factor.

With the end of evaluating the presence of the transgene, a PCR reaction was carried out with a final volume of 25 μL, using the previously described GFLV241 primers. PCR products were visualized by electrophoresis in 1% agarose gels using 0.5×TAE buffer and stained with ethidium bromide under UV light.

As a result, 63 transgenic lines were identified in which the 241 bp fragment of the 388 bp transgene was amplified. These were evaluated by RT-PCR.

### 7. Expression of the Transgene

The expression of the transgene was evaluated by RT-PCR in seedlings that were positive in PCR. Total RNA was extracted using the protocol described by Goes da Silva (2005) with some modifications. Leaves were dissected, placed in an Eppendorf tube and frozen in liquid nitrogen. Then, 2 g of frozen leaves were macerated in a porcelain mortar in the presence of liquid nitrogen until a fine powder was obtained. This was transferred to a 1.5 mL Eppendorf tube and homogenized with 5 volumes of cold sterile extraction solution (200 mM Tris-HCl pH 8.5, 1.5% w/v lithium dodecyl sulfate, 200 mM LiCl, 10 mM/L Na₂EDTA, 1% w/v sodium deoxycholate 1%, 1% v/v NP-40), instantaneously mixed with 2 mM aurin tricarboxylic acid, 200 mM DTT, 10 mM urea and 2% w/v PVPP. Samples were incubated at −80° C. for 24 hours, thawed the next day at 37° C. and centrifuged at 5,000 rpm for 20 min at 4° C. The supernatant was transferred into a clean Eppendorf tube, mixed with 1/30 volume of 3.3 M sodium acetate and ethanol to a final concentration of 10% v/v, incubated on ice for 10 min and centrifuged at 5,000 rpm for 20 min at 4° C. The supernatant was transferred into a clean Eppendorf tube, mixed by inversion with 1/9 volume of sodium acetate and isopropanol to a final concentration of 33% v/v, incubated at −20° C. for 2 hours and centrifuged at 5,000 rpm for 30 min at 4° C. The supernatant was discarded and the precipitate was resuspended with 1 mL of TE buffer (10 mM Tris-HCl pH 7.5 and 1 mM EDTA), incubated on ice for 30 min and centrifuged at 5,000 rpm for 30 min at 4° C. The resulting supernatant was mixed by inversion with ¼ volume of 10 M LiCl and incubated on ice for 24 hours. The next day, it was centrifuged at 10,000 rpm for 30 min at 4° C., the supernatant was discarded and the precipitate was resuspended in 400 μL of TE buffer. Then, 400 μL of 5 M potassium acetate (unadjusted pH) were added, the mix was incubated on ice for 3 hours and centrifuged at 10,000 rpm for 30 min at 4° C. Subsequently, the supernatant was discarded and the resulting precipitate was resuspended in 250 μL of TE buffer. 1:1 phenol:chloroform-isoamylic alcohol (24:1) was added, mixed and centrifuged at 10,000 rpm for 15 min at 4° C. The clean supernatant was transferred into another Eppendorf tube, mixed with 1/9 volume of 3.3 M sodium acetate and 2 volumes of absolute ethanol, incubated at −20° C. for 2 hours and centrifuged at 10,000 rpm for 30 min at 4° C. The supernatant was discarded and the precipitate was washed with 500 μL of absolute ethanol and centrifuged at 10,000 rpm for 10 min. The precipitate was dried at room temperature and resuspended in 50 μL of sterile distilled deionized H₂O. The samples were stored at −20° C.

RNA was quantified according to the same procedure followed for DNA quantitation. RNA concentration was calculated with the formula [ARN] (μg/mL)=Abs₂₆₀ₙₘ×40×dilution factor. The RNA produced was treated with DNase I to remove residual DNA. 1 μL buffer and 2 μL DNase are added to 2 μg RNA, and distilled deionized sterile water is added to a final volume of 10 μL. The mix is then incubated at 37° C. After 1 hour, 1 μL of STOP solution is added and the mix is incubated for 10 min at 65° C. cDNA was obtained through reverse transcription from the obtained RNA using the SuperScript™ II Reverse Transcriptase kit (Invitrogen®). In an Eppendorf tube, 1 μL random primers, 1 μL dNTP mix (10 mM each) and 2 μg total RNA were mixed to a final volume of 12 μL. The mix was heated up to 65° C. for 5 min and rapidly transferred into ice. The tube contents was collected by a short centrifugation and then 4 μL of First-Strand Buffer 5×, 2 μL 0.1 M DTT and 1 μL OUT RNase were added. The contents were mixed and incubated for 2 min at 25° C. Then, 1 μL of SuperScript II Reverse Transcriptase (RT) was added and the mix was incubated for 10 min at 25° C. and then for 50 min at 42° C. The reaction was stopped by incubation for 15 min at 70° C. cDNA integrity was assessed through PCR, by amplifying 300 bp of the constitutive gene G3PDH using the following primers:

Primer 5′-G3PDH:
5′-CGTTCTACTTTCTGGCATCC-3′;
Primer 3′-G3PDH:
5′-GCAAATCGGGTCGTTAATA-3′.

The amplification program consisted of one incubation step at 94° C. for 3 min and 30 cycles of 94° C. for 30 s, 55° C. for 45 s and 72° C. for 1 min. The reaction ended with an extension step at 72° C. for 5 min and cooling to 4° C. Products were visualized in 1% agarose gels as previously described. Then, a second PCR was performed to amplify 80 bp of the transgene with the following primers:

Primer 5′-RT-cp-gflv:
5′-TGGAGAATTGTGTGGTCATGCTA-3′
Primer 3′-RT-cp-gflv:
5′-GCCCGTTAAACACGTAAAATGTAGT-3′.

The PCR reaction consisted of one incubation step at 95° C. for 2 min, 30 cycles of 95° C. for 30 s, 54° C. for 30 s, 72° C. for 45 s, and a final cooling down to 4° C. Products were visualized in 3% agarose gels stained with ethidium bromide under UV light.

The housekeeping G3PDH gene was amplified in all assessed lines. Among these, the 80 bp fragment of transgene cp-gflv was amplified in 26 lines.

### 8. In Vitro GRAFTS

110 Richter seedlings positive for GFLV infection and transgenic seedlings from lines 12, 15, 22, 30, 35 and 60 were cultured in vitro in MS medium at 25° C. with a 16 light hours/8 dark hours photoperiod for 4-5 weeks. GFLV infection positive plants were previously assessed by PCR. To verify the presence of the virus in infected plants, specific primers were used to amplify 1400 bp of the GFLV coat protein, having the following sequences:

Primer C:
5′-CAAGGCAAGTGTGTCCAAA-3′
Primer V:
5′-TGATGCTTATAATCGGATAACTA-3′

Plants that were positive in this reaction were used for in vitro grafts. When transgenic lines developed enough roots, the aerial part was cut off and infected explants were grafted as woody grafts onto transgenic seedlings. After 4-6 weeks, grafts were assessed for the presence of virus.

### 9. Assessment of the Presence or Absence of GFLV in In Vitro Grafts

To evaluate the presence of the virus in grafts, RNA was extracted from frozen leaves as previously described. cDNA was synthesized using the protocol already described and PCR reactions were set up.

The presence of virus in the grafts was assessed by RT-PCR, using primers that amplify 250 bp of the virus movement protein (mv). This was performed this way because the construct has coat protein sequences and their use could lead to wrong results. Primers used in these reactions were:

Primer mv-GFLVpF1:
5′-TTAGTGTTGGCACTTTGCGT-3′
Primer mv-GFLVpR1:
5′ TGATAGAGAAGGTTTGCCCT-3′.

Firstly, cDNA integrity was verified through amplification of the G3PDH constitutive gene, as previously described, and then a PCR was performed with the movement protein primers. Reaction conditions were: 30 cycles of 95° C. for 30 s, 55° C. for 30 s and 72° C. for 30 s.

In 3 of 6 experiments, the PCR product was observed to disappear after 4 or 5 weeks: C3/30, C3/60 and C3/15, where the first number corresponds to the wild type infected line and the second is the transgenic line. In grafts C3/35, C3/12 and C3/22, the presence of movement protein was detected. These results indicate that in 3 of 6 in vitro graft experiments (50%) there was abolition of the GFLV infection, which continued when assessed after 6 months (Table 2).

TABLE 2
Generated transgenic rootstock lines

| Lines with | | | | |
|---|---|---|---|---|
| kanamycin | | Selected RT- | Lines in the | |
| resistance | PCR(+) lines | PCR(+) lines | grafting assay | Assessed lines |
| 86 | 63 | 26 | 6 | 37 |

### 10. Greenhouse Experiments

Finally, grafting experiments were performed in greenhouse conditions. Woody grafts were grafted onto 6˜2 years-old transgenic rootstocks of the invention, each in triplicate. For this aim, non-transgenic wild type plants infected with the virus (GFLV+) were used, which were evaluated by RT-PCR to detect the presence of GFLV. After 3 months of growth, the presence of virus in the grafts was determined by RT-PCR using the primers described in Example 9. As expected, a complete infection abolishment was detected, as well as the disappearance of the characteristic symptoms in the graft leaves, in the same lines evaluated in vitro. FIG. 2 shows the result of the RT-PCR for one of the grafts of the invention (graft 28) that was grafted with GFLV-infected plant 1(i28/1), after 3 months of growth. The first lane contains cDNA from graft i28/1, the second lane is the (RT) control for the same graft (i28/1 (RT−)), the third lane contains cDNA from the GFLV-infected plant 1 (GFLV+1) and the last lane is the negative PCR control. The Figure shows that the original GFLV+1 plant transcribes the virus movement protein, whereas in the graft i28/1 this transcript disappears. This demonstrates that the rootstock of the invention allows sanitation of a plant infected with the grapevine fanleaf virus (GFLV).

1. A vector plasmid that confers resistance and sanitation against the grapevine fanleaf virus (GFLV), wherein said vector plasmid contains SEQ ID No 1 and SEQ ID No 2 spaced apart by an intron.

2. A vector plasmid according to claim 1, wherein said vector plasmid further contains a gene conferring antibiotic resistance.

3. A vector plasmid according to claim 2, wherein said vector plasmid comprises the neomycin phosphotransferase II (nptII) gene conferring kanamycin resistance.

4. A transformed plant cell wherein said transformed plant cell contains and expresses the vector plasmid of claim 1.

5. A transformed plant cell according to claim 4, wherein said transformed plant cell is an embryogenic cell in globular state.

6. A transformed plant cell according to claim 4, wherein said transformed plant cell is a grapevine cell.

7. A transformed plant cell according to claim 6, wherein said transformed plant cell is a cell of one of the following grapevine varieties: 100 Richter; Freedom or Harmony.

8. A transformed plant cell according to claim 7, wherein said transformed plant cell is a cell of the 100 Richter grapevine variety.

9. A method to confer resistance and sanitation against the grapevine fanleaf virus (GFLV) in non-transgenic grapevines, wherein said method comprises the steps of:
a. providing a group of plant cells transformed with a vector plasmid according to claim 4;
b. culturing the group of transformed plant cells to form transgenic seedlings resistant to the grapevine fanleaf virus (GFLV);
c. culturing said transgenic seedlings to take roots;
d. cutting the aerial part of the transgenic seedlings;
e. grafting a non-transgenic grapevine woody graft onto said seedling; and
f. culturing the graft
wherein the non-transgenic grapevine plant acquires resistance and sanitation against the grapevine fanleaf virus (GFLV) from the phloem of the transgenic plant.

10. A method according to claim 9 wherein said grafted non-transgenic grapevine is Vitis vinifera.

11. A method according to claim 10 wherein the grafted non-transgenic grapevine is a variety of Vitis vinifera selected among the following table grape varieties: Autumn royal, Black seedless, Calmeria, Emperor, Flame seedless, Loose Perlette, Red Malaga, Ruby seedless, Loose Perlette, Thompson seedless, Red Globe, Sugarone and Superior seedless.

12. A method according to claim 10 wherein the grafted non-transgenic grapevine is a variety of Vitis vinifera selected among the following wine grape varieties: Carmenère, Cabernet sauvignon, Cabernet Franc, Syrah, Chardonnay, Courdec, Dattier, Emerald, Malbec, Merlot, Mission, Muscat, Pinot noir, Riesling, Sauvignon blanc, Sémillon, Shiraz, Tempranillo, Zinfandel.
